(19) United States
(12) Patent Application Publication
(10) Pub. No.: US 2008/0140187 A1
(43) Pub. Date: Jun. 12, 2008

(54) Anti-clotting, anti-microbial, anti-inflammatory medical stent

(76) Inventors: Arthur A. Krause, Hollywood, CA (US); Walter K. Lim, Rancho Santa Fe, CA (US)

Correspondence Address: Dennis H. Lambert, 7000 View Park Drive, Burke, VA, 22015, US

(21) Appl. No.: 11/975,358

(22) Filed: Oct. 18, 2007

Related U.S. Application Data
(63) Continuation-in-part of application No. 11/252,182, filed on Oct. 17, 2005.
(60) Provisional application No. 60/619,233, filed on Oct. 15, 2004.
(60) Provisional application No. 60/701,897, filed on Jul. 22, 2005.
(60) Provisional application No. 60/852,597, filed on Oct. 18, 2006.

(51) Int. Cl. A61F 2/82

(52) U.S. Cl. 623/1.43

(57) ABSTRACT

A medical stent includes an open-ended cylindrical body movable between a collapsed position for insertion into a body lumen and a radially expanded position pressed against the wall of the lumen. At least a portion of the stent is made of a material having anti-clotting and/or antimicrobial properties. In one embodiment, concentric laminated tubes of dissimilar materials such as copper and silver form the body. In another embodiment stacked rings of different materials such as, e.g., copper, silver, and/or steel form the body. In a still further embodiment at least one attachment made of a material having anti-clotting or antimicrobial properties is secured to the stent body. In a further embodiment the body has a lattice-like sidewall with openings therethrough, and a cover is attached to the outer surface. In all forms the body may have an outwardly flared inlet end to reduce turbulence.

This application claims the benefit of prior copending U.S. provisional patent application Ser. No. 60/852,597, filed Oct. 18, 2006, and is a continuation-in-part of application Ser. No. 11/252,182, filed Oct. 17, 2005, which, in turn, claims the benefit of U.S. provisional patent application Ser. No. 60/619,233, filed Oct. 15, 2004, and Ser. No. 60/701,897, filed Jul. 22, 2005.

### TECHNICAL FIELD

The present invention relates to medical devices. In particular, the present invention relates to stents for placement in a body lumen to correct or treat a diseased area in the lumen.

### BACKGROUND ART

Diseased tissue generally is treated with surgical intervention, or drug therapy, or a combination of both. The therapeutic alternatives available for treatment of vascular disease, for example, which is caused by progressive blockage, or stenosis, of the blood vessels that perfuse the heart and other major organs, normally include surgical intervention to remove the blockage, i.e., replacement of the blocked segment with a new segment of artery, or the use of a catheter-mounted device such as a balloon catheter to dilate the artery. Both procedures are medical procedures whose purpose is to increase blood flow through an artery.

Inflation of a balloon to dilate the artery is known as angioplasty and is the predominant treatment for vessel stenosis. The increasing use of this procedure is attributable to its relatively high success rate and its minimal invasiveness compared with coronary bypass surgery. During angioplasty, a balloon catheter in a deflated state is inserted within a stenotic segment of a blood vessel and inflated and deflated one or more times to expand the vessel by compressing the built-up tissue or plaque in the vessel lumen to enlarge the opening and restore blood flow.

Angioplasty often permanently opens previously occluded blood vessels. However, a limitation associated with angioplasty is the abrupt closure of the vessel that may occur immediately after the procedure, and restenosis, which occurs gradually following the procedure and refers to the re-narrowing of an artery after an initially successful angioplasty. Restenosis is also a chronic problem in patients who have undergone saphenous vein bypass grafting. Post-angioplasty closure of the vessel, both immediately after angioplasty (acute reocclusion) and in the long term (restenosis), is a major difficulty associated with angioplasty.

Because 30-50% of patients undergoing angioplasty will experience restenosis, the success of angioplasty alone is clearly limited as a therapeutic approach to coronary artery disease. Accordingly, stents of various configurations have been developed and used to hold the lumen of a blood vessel open following angioplasty. Balloon angioplasty and associated implantation of a stent or stents compress the built-up tissue or plaque in a vessel lumen to enlarge the opening and restore blood flow. There is a multiplicity of different stents that may be utilized following angioplasty. Examples are disclosed in U.S. Pat. Nos. 5,766,710, 6,254,632, 6,379,382 and 6,613,084, and in published US applications 2002/0062147, 2003/0065346, 2003/0105512, 2003/0125800, 2003/0181973, 2003/0225450 and 2004/0127977. Most stents are compressible for insertion through small cavities, and are delivered to the desired implantation site percutaneously via a catheter or similar transluminal device. Once at the treatment site, the compressed stent is expanded to fit within or expand the lumen of the passageway.

Stents typically are made of a suitable metal such as stainless steel, and generally are either self-expanding or are expanded by inflating a balloon positioned inside the compressed stent at the end of the catheter. Stents as initially developed and still in common use consisted essentially of the metal from which they are made and are referred to as bare metal stents, i.e., the bare metal of the stent is exposed to body tissue

Stenting with bare metal stents, however, may not always be successful because small muscle cell (SMC) proliferation and migration are intimately involved with the pathophysiological response to arterial injury. That is, once a stent is implanted, the blood vessel grows new layers of cells over the metal as part of the healing process. However, whenever any foreign body comes into contact with the blood, there is a propensity for clots to form. The stent is recognized as a foreign body prior to being masked by cells forming over it, and as part of the healing process the body sends a clotting protein to the stented site, potentially causing the formation of blood clots. Problems also arise when the healing is too vigorous and the resulting layer of cells becomes too thick and obstruct or partially obstruct the lumen.

To solve the problem of excessive tissue growth around the bare metal stent, researchers initially turned to the use of radioactive seeds planted in blood vessels for a short time following implantation of a stent. The radioactive material slowed or impeded the growth of new cells during the healing process, thus preventing restenosis. However, this approach only delayed restenosis and potentially damaged the vessel, leading to other ills such as clots.

Researchers next turned to drug eluting stents to slow the healing process without the problems associated with the use of radioactive seeds, and also to eliminate or minimize the formation of blood clots. The therapeutic substances typically are either impregnated into the stent or carried in a polymer that coats the stent and are released from the stent or polymer once it has been implanted in the vessel.

The local delivery of drug/drug combinations from a stent is advantageous because the scaffolding action of the stent prevents vessel recoil and closure, while the drug or drugs delivered from the stent prevent multiple components of neointimal hyperplasia or restenosis. This local administration of drugs, agents or compounds to stented arteries may also have additional therapeutic benefit. For example, higher tissue concentrations of the drugs, agents or compounds may be achieved utilizing local delivery, rather than systemic administration. In addition to maintaining higher tissue concentrations of a drug or drug combination, local delivery reduces systemic toxicity compared with systemic administration. Also, in utilizing local delivery from a stent rather than systemic administration, a single procedure may suffice with better patient compliance. An additional benefit of combination drug, agent, and/or compound therapy may be to reduce the dose of each of the therapeutic drugs, agents or compounds, thereby limiting their toxicity, while still achieving a reduction in restenosis, inflammation and thrombosis. Local stent-based therapy is therefore a means of improving the therapeutic ratio (efficacy/toxicity) of anti-restenosis, anti-inflammatory, and anti-thrombotic drugs, agents or compounds. Substances that are commonly delivered from stents are identified in applicant's copending provisional application Ser. No. 60/852,597 and in U.S. Pat. No. 6,379,382, the disclosures of which are incorporated in full herein

However, in some studies no new protective layer of cells formed over the drug eluting stent even months after its implantation. As a result, the body still viewed the site of the stent as an unhealed wound and sent a clotting protein to speed healing. This, in turn, can lead to formation of a deadly clot. Also, drug eluting stents are generally limited in their capacity to carry therapeutic drugs, especially when more than one pathophysiological condition needs to be treated, or treatment needs to be carried out over a prolonged period of time. Applicant's stents as described and claimed in copending application Ser. No. ______ solve some of these problems by providing extra drug carrying capacity, but the stents may still require the use of drugs to control clotting.

Efforts have been made to prevent the formation of clots by incorporating clot preventing material in the stent. For example, it is known that copper ions break down or catalyze nitrosothiols in the blood to produce nitric oxide, which relaxes blood vessels, increases blood flow, and prevents clot-forming platelets from attaching to implant surfaces. Stents have therefore been developed with thin polymer coatings that release nitric oxide directly to prevent clot formation on implants. However, because the polymer coatings are extremely thin, only a limited supply of nitric oxide can be carried. In an effort to solve this problem, rather than incorporating nitric oxide in the polymer coating and releasing it directly, researchers incorporated copper ions in the polymer coating to catalyze the breakdown of nitrosothiols, thereby stimulating the local production of nitric oxide. This prolonged the effective life of the stent, but it was still limited due to the limited supply of copper ions that can be incorporated in the thin polymer coating.

Further, both bare metal and drug eluting stents can induce turbulence in the blood flow and produce stagnant pools of blood, potentially resulting in the formation of clots.

Thus, it would be advantageous to provide a stent made of a material that prevents the formation of clots, and that is constructed to avoid or minimize stagnation or pooling of blood at the stented site.

### DISCLOSURE OF THE INVENTION

The stent in accordance with the invention is made of a material that inhibits the formation of blood clots at the stented site, and is constructed to minimize turbulence and formation of stagnant pools of blood flowing through the stent. The stent includes an open-ended cylindrical body carried on a distal end of a catheter for insertion into the body lumen and placement at the stenosed site. The cylindrical body is movable between a collapsed position for insertion into the body lumen, and a radially expanded position pressed against the wall of the body lumen.

The stent of the invention preferably either is made from copper or a copper alloy, or is made from a conventionally used material and has copper or copper alloy attachments secured to it, or is plated or coated with copper. Copper ions released by the copper react with the nitrosothiols in blood to stimulate the production of nitric oxide, thereby preventing the formation of clots at the stented site. The relatively large amount of copper ions available in the stent of the invention as compared with prior art devices results in a stent that effectively can release copper ions as long as the stent is in place.

The stent body can have any suitable construction, including, for example, a series of oppositely diagonally extending struts interconnected at crossing points to form a lattice-like structure, or a slotted tube, or a series of longitudinally spaced circumferentially extending zigzag-shaped elements interconnected by longitudinally extending members, or any of the structures employed in the prior art and/or in applicant's prior copending application Ser. Nos. 60/852,597 and 11/252,182.

In one embodiment of the stent of the invention, the stent body can be formed of spirally wound bands or ribbons, preferably made of copper, interconnected at their opposite ends and slightly spaced apart at adjacent side edges. In a preferred construction the bands spiral through about 1.5 turns from one end of the stent to the other, but a different number of turns could be negotiated by the bands. This construction imparts a swirl motion to blood flowing through it, while at the same time presenting a relatively smooth interior surface to the blood flow, thereby helping to prevent formation of stagnant pools of blood without imposing turbulence or shear stresses on the blood.

In another embodiment of the invention, different parts of the stent body are constructed of dissimilar metals and/or other materials selected for their different properties. In one construction, the different materials are exposed at different portions of the stent body. In one form of this embodiment, the different materials are incorporated in different layers or laminations that are formed into concentric tubes and then cut with a laser or other suitable means to form the lattice structure of the stent, with one material exposed at the inner surface of the stent body and another material exposed at the outer surface. For example, an intermediate layer could comprise stainless steel, selected for its strength, an outer layer could comprise copper, selected for its therapeutic properties, and an inner layer could comprise another material selected for its particular properties. In another form of this embodiment, the concentric tubes can be formed with segments or strips of different materials, each extending over the length of the stent but over only part of the circumference of the stent body, whereby not only can different materials be exposed at the inner and outer surfaces of the stent, but different materials can be exposed at different locations around its circumference. In a further form of this embodiment, rings of different materials are stacked and sonic or spot welded to each other to form a tubular structure, with different materials exposed along the length of the stent. Any number of rings can be employed, wherein succeeding rings along the length of the stent may comprise, for example, silver, steel, copper; silver, steel, copper; silver, steel, copper, and so on.

Separate attachments made of copper, gold, and/or silver, and/or alloys thereof, can be applied to conventional stent bodies. The attachments can take various forms, including: beads of the selected metal, such as copper, gold, and/or silver, or alloys thereof, welded in place; or rivets attached at selected locations; or small pads or discs or other structures bonded to the stent body by the application of high pressure; or secured by mechanical locking or clamping, e.g., folded tabs or edges on the attachments; or applied as a coating applied by electroplating or sputtering and the like. A strategic location of the attachments could be near the forward or leading end of the stent, or the attachments can extend throughout the length of the stent. The attachments can also take the form of the covers or plates that expand as the stent is expanded.

The stent of the invention can be made of materials other than copper and also having beneficial properties, such as, for example, gold, silver and/or platinum, or alloys thereof, including alloys of these metals with copper. Silver, for example, is known to have antimicrobial properties and to promote healing, and a stent made from silver or a silver alloy, or coated or plated with silver or a silver alloy, or having silver or silver alloy attachments affixed to it, would reduce or avoid inflammation and promote healing at a stented site. Silver could be alloyed with copper, for example, to derive the benefits of both. Gold and silver both have anti-clotting properties, and could be used in lieu of or in combination with other metals.

Each of the forms may be adapted in a manner as discussed in the following paragraphs to carry a drug or drugs on its outer surface.

For instance, if the stent body is formed by a plurality of interconnected struts or elements forming a lattice structure having openings therethrough, a plurality of enlarged pads or depots can be placed or formed at the intersections of at least some of the struts for carrying a therapeutic agent, or different therapeutic agents on different pads. The drug or drugs may be held in holes formed through the pads, or in depressions or a roughened surface formed in the surface of the pads, or in other ways known in the art, such as in a polymer coating on the pads, and the like. Roughened surfaces or holes or depressions for carrying drugs may also be formed in the bands, ribbons, covers and plates employed in other forms of the invention.

The various forms of drug eluting stent according to the invention avoid the problems associated with prior art stents, wherein the drug or drugs are placed in openings or depressions formed in the stent structure itself, thus weakening the stent structure, or are carried either directly on the stent body or imbedded in a polymer substrate coated on the stent body and thus subject to dislodgement as the stent body expands during implantation.

The stent body in any or all of the forms of the invention may be coated with Teflon on at least its inner surface. One of the advantages of Teflon-coating of the stent is to ease blood flow through the stent channel. Additionally, adherence of blood platelets to the inner walls of the stent will be resisted. Coating of the stent body with Teflon is possible in various embodiments of the present invention because the plates, ribbons and pads attached to the outer surface of the stent body in those embodiments can carry the drug or drugs. Obviously, when the stent is coated with Teflon a drug or copolymer for carrying the drug cannot be adhered to the stent body, as in conventional stents.

In all of the preceding embodiments, and especially the swirl-inducing embodiment, the stent body may have a slightly outwardly flared inlet end. It has been noted in many studies that as the blood flows through the vascular tunnel and hits the opening or beginning of an implanted stent, the end of the stent may disturb the flow of blood and cause stagnation, shear stress, and/or turbulence at this point. It may also cause disturbance of the blood flow as it passes through the vascular channel downstream of the stent. The slightly outwardly flared inlet end of the stent in this embodiment effectively reduces or eliminates this disturbance and prevents stagnation, shear stress, and/or turbulence caused by the stent.

The ribbons, plates, covers, laminated tubes, stacked rings, and stents themselves in the various embodiments described above can be made of copper or copper alloys, and/or other materials such as silver, steel, zinc, chrome, carbon, gold, brass, tantalum, titanium, platinum, sulfur compounds, and/or alloys or compositions thereof that produce the desired results.

Various therapeutic substances can be applied in any desired manner and combination to the auxiliary structures, i.e., to the ribbons and plates that are attached to the outside of the stent body in accordance with the present invention, or to the laminated tubes, stacked rings, or bands forming the stent bodies. In one embodiment the agents are provided only in spaced areas so that the material of the underlying structure is exposed between the spaced areas. The exposed areas can thus provide or produce additional biological or pharmacological benefit. For example, if the underlying structure is made of copper or silver it can impede or prevent restenosis through the production of, e.g., copper ions that catalyze the breakdown of blood chemicals to produce nitric oxide, as discussed above. If copper ions are relied upon in this manner as a preventative for stenosis and restenosis, then it would not be necessary to put drugs or medications on the stent for this same purpose.

The therapeutic substances can comprise, for example, anticoagulants, antiplatelets, and cytostatic agents. Compounds such as Lecithin, Allicin (a raw garlic extract) and/or onion extracts, and HDL, are examples of naturally occurring substances that can be used. Other examples include those identified in U.S. Pat. No. 6,379,382, the disclosure of which is incorporated herein, and heparin and heparin fragments, colchicine, taxol, angiotensin converting enzyme (ACE) inhibitors, angiopeptin, and cyclosporin A. These substances are exemplary only, and are not intended to be limiting on the present invention.

### BRIEF DESCRIPTION OF THE DRAWINGS

The foregoing, as well as other objects and advantages of the invention, will become apparent from the following detailed description when taken in conjunction with the accompanying drawings, wherein like reference characters designate like parts throughout the several views, and wherein:

FIG. 1 is a side view in elevation of a stent according to the invention, wherein the stent body comprises intersecting struts that form a lattice-like structure.

FIG. 2 is a side view in elevation of a second form of stent according to the invention, wherein the stent body comprises a slotted tube.

FIG. 3 is a side view in elevation of a third form of stent according to the invention, wherein the stent body comprises a series of longitudinally spaced, circumferentially extending zigzag structures interconnected by longitudinal elements.

FIG. 4 is a greatly enlarged fragmentary plan view of a portion of a stent comprising woven strands according to an embodiment of the invention.

FIG. 5 is a side view of the woven structure of FIG. 4.

FIG. 6 is an exploded perspective view depicting several sheets of material in position to be laminated together in a multi-layered structure or substrate for use in forming a stent of generally tubular configuration.

FIG. 7 is a transverse sectional view of several layers of material laminated together to form a sheet used in forming a tubular stent.

FIG. 8 is a perspective view of an example of a stent that can be made using multiple layers of material laminated together.

FIG. 9 is a longitudinal sectional view of the stent of FIG. 8.

FIG. 10 is a side view in elevation of a sixth form of the invention, wherein the stent comprises a plurality of slightly spaced apart spirally wound bands extending along the length of the stent to impart a swirling motion to blood flowing through the stent.

FIG. 11 is a slightly enlarged fragmentary view in side elevation showing how the inlet end of the stent according to any of the foregoing forms of the invention can be outwardly flared to facilitate smooth flow of blood entering the stent.

FIG. 12 is an exploded view of rings of dissimilar materials that may be stacked together to form a tubular stent body.

FIG. 13 is a side view in elevation of a stent body formed of stacked rings of dissimilar materials so that different materials are exposed along different parts of the length of the stent.

FIG. 14 is a perspective view of a stent body formed of plural layers of strips of dissimilar materials so that different materials are exposed at the inner and outer surfaces of the stent and at different circumferential portions of the stent.

FIG. 15 is a view in side elevation of a stent body comprising interconnected strut elements forming a lattice-like stent structure, wherein the strut elements are comprised of different materials in different zones along the length of the stent.

FIG. 16 is an end view of a stent formed of three concentric layers of different materials, including an outer layer of copper, an intermediate layer of steel, and an inner layer of silver.

FIG. 17 is an end view of a stent formed of two concentric layers of different materials, including an outer layer of copper and an inner layer of steel.

FIG. 18 is a side view in elevation of a tubular stent body according to one of the forms of the invention shown in FIG. 16 or 17, prior to being cut to form a lattice-like structure.

FIG. 19 is a fragmentary side sectional view of a stent body according to FIG. 16.

FIG. 20 is a side view in elevation of a stent formed of interconnected strut elements and having enlarged pads at some of the intersections for carrying a drug or drugs.

FIG. 21 is an enlarged fragmentary view in side elevation of a stent having ribbons attached to it in accordance with one form of the invention, wherein the ribbons extend longitudinally of the stent in generally straight, parallel relationship to one another.

FIG. 22 is an enlarged fragmentary view in side elevation of a second embodiment of stent having ribbons attached to it in accordance with the invention, wherein the ribbons are wound around the stent in a spiral pattern, extending longitudinally of the stent in generally parallel relationship to one another.

FIG. 23 is an enlarged fragmentary view in side elevation of a third embodiment of stent having ribbons attached to it in accordance with the invention, wherein the ribbons are applied to the stent in a zigzag pattern, extending longitudinally of the stent in generally parallel relationship to one another.

FIG. 24 is a greatly enlarged fragmentary plan view of a portion of a ribbon in any of the forms of the invention shown in FIGS. 21-23, wherein intermittent, spaced roughened or textured areas are formed on it for holding a drug or other beneficial agent.

FIG. 25 is a greatly enlarged fragmentary plan view of a portion of a ribbon having intermittent, spaced openings or holes formed through it for holding a drug or other beneficial agent.

FIG. 26 is a taken along line 26-26 in FIG. 25.

FIG. 27 is a greatly enlarged longitudinal sectional view similar to FIG. 26, of a ribbon having intermittent, spaced recesses or depressions formed in it for holding a drug or other beneficial agent.

FIG. 28 is a greatly enlarged longitudinal sectional view similar to FIG. 26, of a ribbon having intermittent, spaced beads of copper or a copper alloy deposited on its surface.

FIG. 29 is a greatly enlarged longitudinal sectional view similar to FIG. 26, of a ribbon having intermittent, spaced rivets of copper or a copper alloy affixed to it.

FIG. 30 is a perspective view of a second form of the invention incorporating a cover, wherein a coiled cover is attached to and covers the stent.

FIG. 31 is an enlarged end view of the device of FIG. 30, depicting the manner in which the coiled cover is attached to the stent, shown with the stent collapsed or crimped to its contracted condition after manufacture.

FIG. 32 is a perspective view of the device of FIG. 30, showing the stent and cover in their expanded condition.

FIG. 33 is a side view in elevation of a third form of the invention incorporating a cover, similar to that shown in FIG. 32, but wherein plural coiled covers are arranged end-to-end along the length of the stent.

FIG. 34 is an end view of the device of FIG. 32 or 33.

FIG. 35 is a perspective view of a first form of the invention incorporating an expandable cover, wherein a longitudinally pleated cover is attached to and covers the stent.

FIG. 36 is an enlarged end view of the device of FIG. 35, depicting the manner in which the pleated cover is attached to the stent, shown with the stent collapsed or crimped to its contracted condition after manufacture, and with the cover shown in an exaggerated, partially unfolded state.

FIG. 37 is a perspective view of the device of FIG. 35, showing the stent and cover in their expanded condition.

FIG. 38 is an end view of the device of FIG. 37.

FIG. 39 is a side view in elevation of a fourth form of the invention incorporating a cover, wherein circumferentially overlapped plates are attached to the stent body, similar to fish scales, with the device shown in its collapsed condition.

FIG. 40 is a plan or developed view showing how the plates of FIG. 39 are overlapped.

FIG. 41 is a plan or developed view showing how the plates of FIG. 40 are related to one another when the stent is in its expanded condition.

FIG. 42 is a plan view of one of the plates that can be used in the form of the invention shown in FIG. 39, wherein the plate has a plurality of holes or depressions formed in it for attaching a drug or drugs to the plate.

### BEST MODES FOR CARRYING OUT THE INVENTION

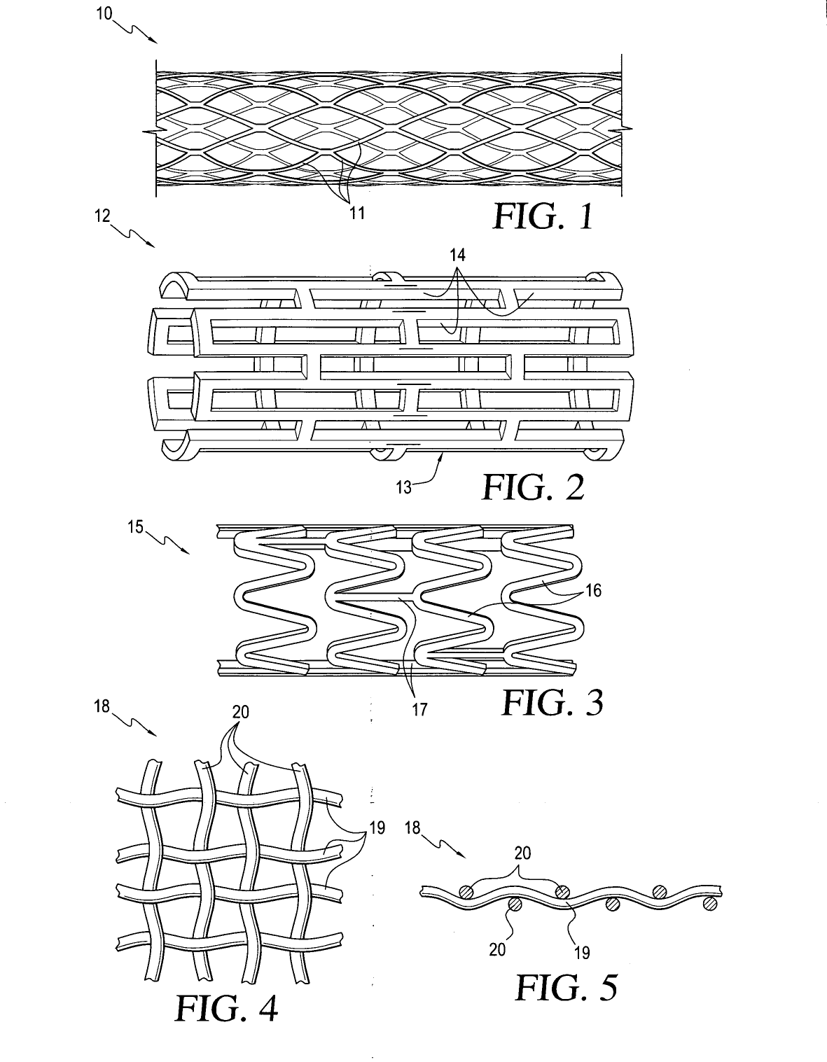

A first embodiment of a stent body suitable for use in the invention is indicated generally at 10 in FIG. 1. This stent body is of conventional construction in that it comprises a series of interconnected struts 11 forming a lattice-like structure. However, in accordance with the invention the stent body is made from an anti-clotting and/or antimicrobial material, such as copper and/or silver and/or alloys thereof. Copper, for example, as discussed previously, can release copper ions to react with nitrosothiols in the blood to produce nitric oxide, an anti-clotting agent.

A second embodiment of stent body suitable for use in the invention is indicated generally at 12 in FIG. 2. This stent body is of conventional construction in that it comprises a tube 13 that is slotted at 14 to form an expandable body having openings through it. However, in accordance with the invention the stent body is made from an anti-clotting and/or antimicrobial material, such as copper and/or silver and/or alloys thereof.

A third embodiment of stent body suitable for use in the invention is indicated generally at 15 in FIG. 3. This stent body is of conventional construction in that it comprises a series of longitudinally spaced, circumferentially extending zigzag structures 16 interconnected by longitudinal elements 17 to form an expandable body having openings through it. However, in accordance with the invention the stent body is made from an anti-clotting and/or antimicrobial material, such as copper and/or silver and/or alloys thereof.

A fourth embodiment of stent body suitable for use in the invention is indicated generally at 18 in FIGS. 4 and 5. The stent body in this form of the invention comprises a woven structure of generally longitudinally extending elements 19 and generally circumferentially extending elements 20. The elements 19 and 20 may comprise the same or dissimilar materials, such as copper and/or silver and/or gold and/or alloys of these and other metals selected for their desired properties.

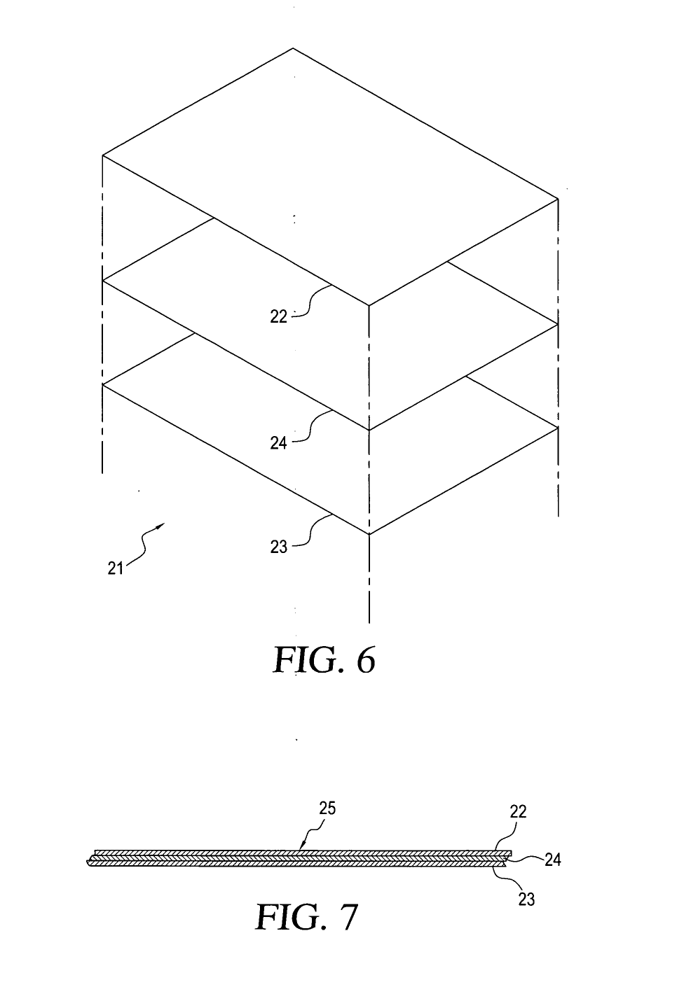

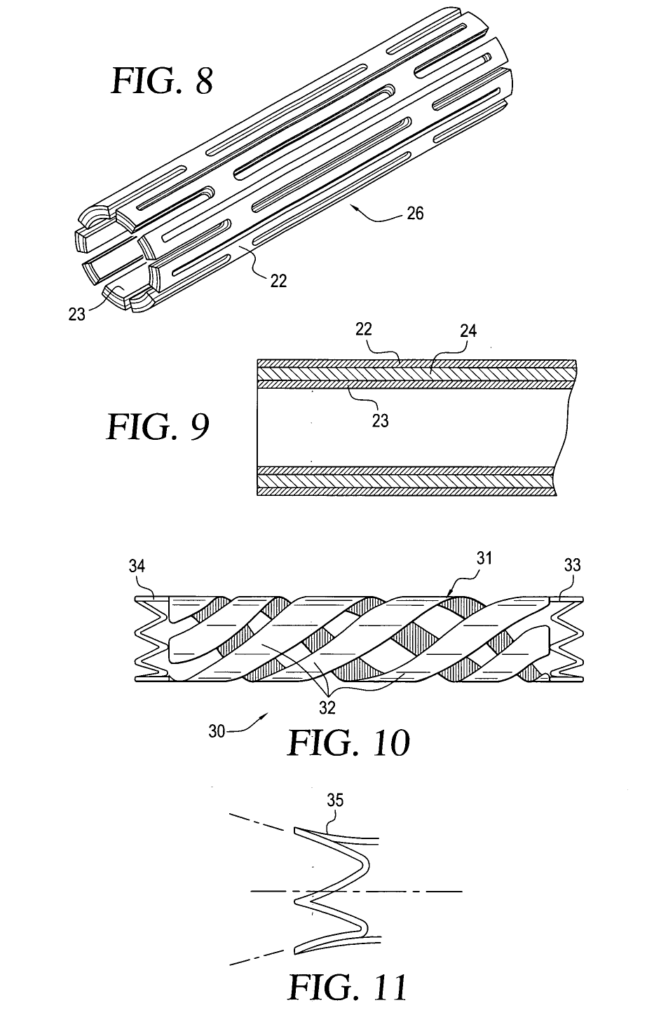

A fifth embodiment of the invention is indicated generally at 21 in FIGS. 6-9. In this form of the invention, a plurality of layers of one or more materials 22, 23 and 24 may be laminated together to form a sheet 25 that can then be formed into a tubular structure and cut with a laser or other known process to produce a stent such as shown at 26, for example, with layer 23 exposed to the blood on the interior of the stent. Layer 23 can be formed of copper to prevent the formation of clots on the stent. Layer 22 would be exposed to the vessel wall and can comprise silver, or gold, or copper, or other material selected for its properties. Layer 24 can comprise stainless steel, for example, for its strength. Lamination of the layers may be accomplished in accordance with conventional processes, e.g., they may be cold-pressed together under sufficient pressure to fuse, the layers together, or the layers may be welded together, etc. Similarly, forming of the tubular structure and cutting it into a desired design can be accomplished using known processes and techniques, such as by cutting with a laser. One or more of the layers can be produced by ion deposition, or by powder coating, or other processes for coating one material onto another.

A sixth embodiment of the invention is indicated generally at 30 in FIG. 10. In this form of the invention the stent body 31 is formed of a plurality of spirally wound, slightly spaced apart bands 32 interconnected at their proximal and distal ends 33 and 34. The bands induce a swirling motion to blood flowing through the stent, thereby preventing stagnation of the blood, but also present a relatively smooth interior surface to the flow of blood.

As shown in FIG. 11, the inlet end 35 of the stent can be slightly outwardly flared to smooth the flow of blood entering the stent and prevent turbulence and shear at this point, aiding in the initiation or transition to a swirling motion in the flow of blood entering the stent. A flared end can be applied to any of the forms of stent disclosed herein.

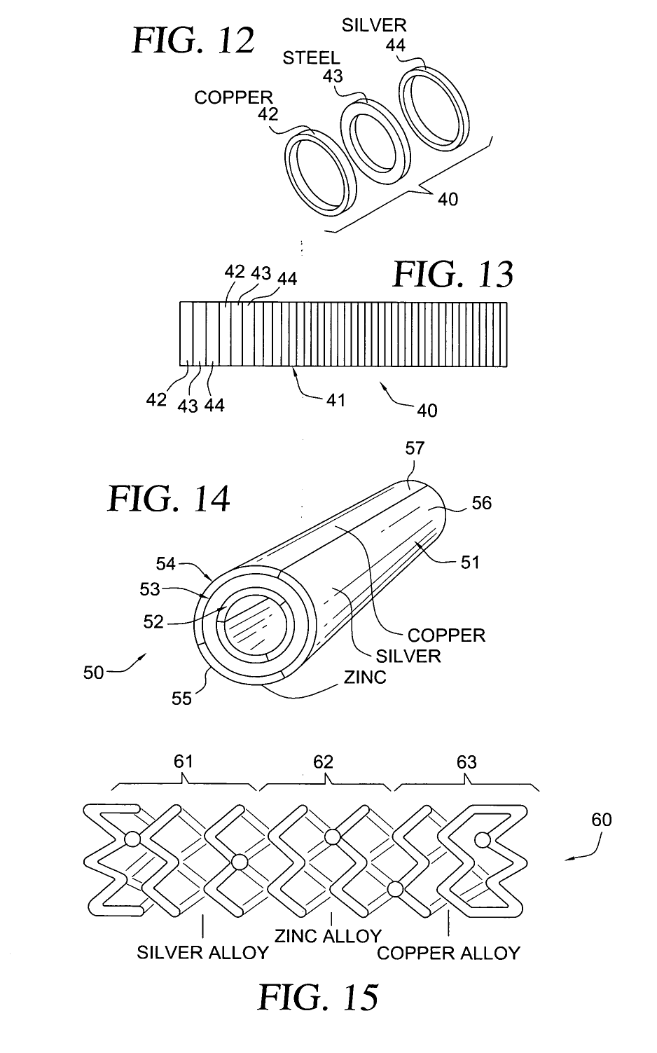

A seventh embodiment of the invention is indicated generally at 40 in FIGS. 12 and 13. In this form of the invention, the stent body 41 is formed of stacked rings 42, 43, 44, etc. . . . , secured together as by welding or the like to form a hollow tubular structure. The rings preferably comprise dissimilar materials, such as alternating rings of copper, steel and silver. It should be understood that any desired and suitable material could be used for the rings to accomplish the objectives of the present invention.

An eighth embodiment is indicated generally at 50 in FIG. 14. In this form, the stent body 51 is formed of laminated concentric tubes 52, 53 and 54 each made up of strips or panels 55, 56, 57 of dissimilar materials secured to each other along longitudinal edges and extending the length of the stent body. As shown, the strips or panels extend axially of the stent, but they could extend in a spiral or other shape, if desired (not shown). The material of the inner and outer layers or laminations 52 and 54 can be selected for any therapeutic property they may have (e.g., copper, gold, silver, etc.) in accordance with the objectives of the present invention, and the intermediate layer can be selected for strength (e.g., steel, chrome, etc.).

A ninth embodiment is indicated generally at 60 in FIG. 15, wherein different axial segments 61, 62 and 63 of the stent body (shown here as an open lattice design) are formed of different materials. In the specific example shown, one end segment 61 is made of a silver alloy, the center segment 62 is made of a zinc alloy, and the second end segment 63 is made of copper or a copper alloy. The different materials are selected for their different properties in accordance with the objectives of the present invention.

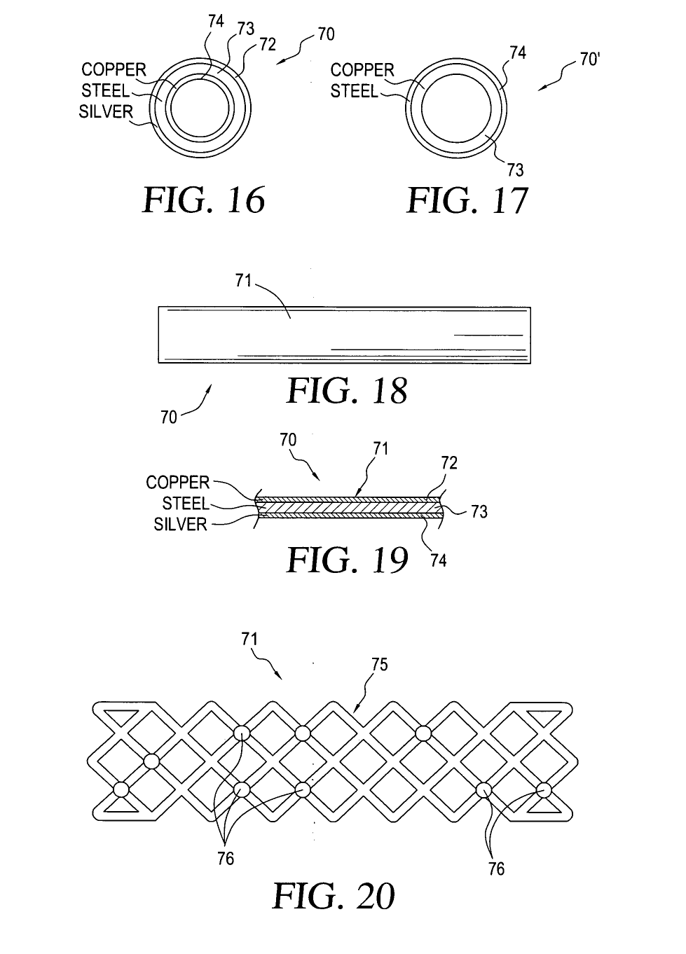

A tenth embodiment is indicated generally at 70 in FIGS. 16, 17, 18 and 19. In this embodiment the tubular stent body 71 is formed of laminated together concentric tubes 72, 73, 74 of different materials, as in the FIG. 14 embodiment, but the concentric tubes each comprise a single material rather than the panels or strips of the earlier embodiment. FIG. 17 shows an alternate form 70′ wherein only two layers 73 and 74 are used to form the tubular structure. In these forms of the invention, the same material would be exposed along the circumference and length of the stent, but different materials would be exposed at the inner and outer surfaces. FIG. 18 shows the tubular stent body before it is cut to form the open lattice-like structure, and FIG. 20 shows the stent body 75 after it is cut. As shown in FIG. 20, enlarged pads or depots 76 can be formed on the stent at selected intersections of the strut elements. The enlarged pads can function to expose a greater extent of copper, and/or at least some of them can carry a drug or drugs for a desired therapeutic benefit.

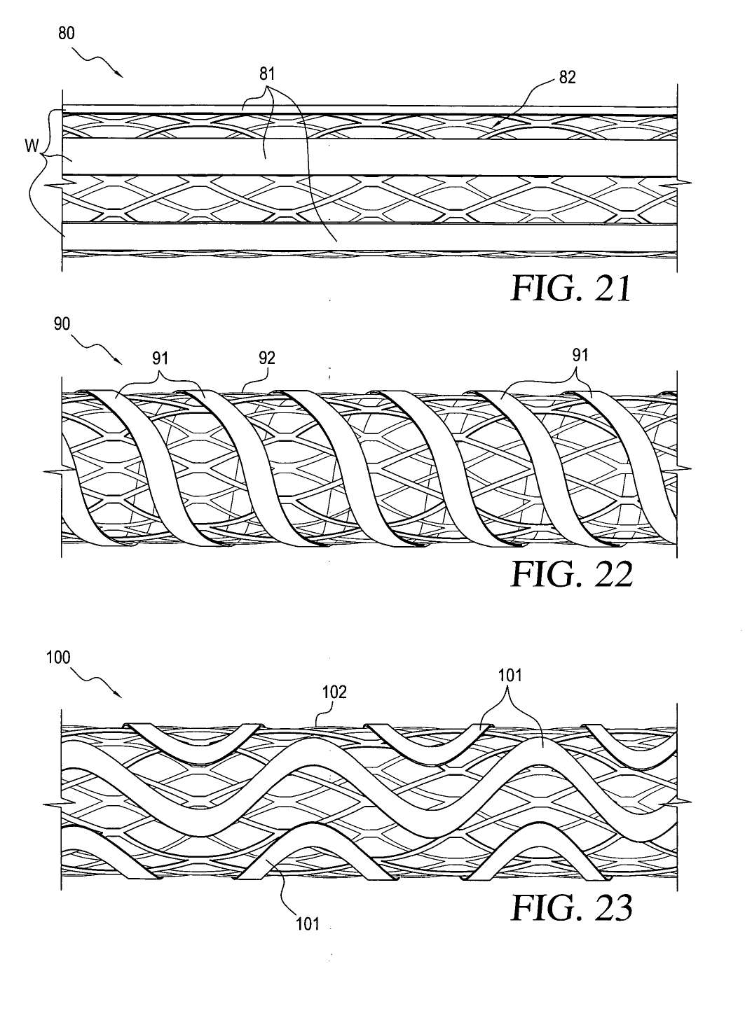

An eleventh embodiment of a stent with auxiliary treatment structure according to the invention is shown generally at 80 in FIG. 21. In this embodiment, a plurality of relatively wide bands or ribbons 81 are attached at least at one end to one end of the stent 82, and extend generally straight and parallel to one another longitudinally of the stent. The bands are made of copper or a copper alloy, or other material selected for its properties in accordance with the present invention. The stent may be of any suitable construction, and in the example shown is of the type depicted in FIG. 1.

In order to permit expansion of the stent, the ribbons preferably are attached to the stent at only one end. In some stent constructions, the ribbons may be attached to both ends of the stent, and when the stent is expanded radially, it can shrink axially to accommodate expansion, even with the ribbons attached to both ends of the stent. Attachment of the ribbons can be by welding or other means known in the art, as represented at W in FIG. 21. Although not shown, it should be understood that the following embodiments could be similarly secured.

A twelfth embodiment is shown at 90 in FIG. 22, wherein the ribbons 91 are wound around the stent 92 in a spiral pattern. As in the previous form, the ribbons can be attached at only one end or at both ends, depending upon the structure of the stent and the ability of the stent to undergo radial expansion with the ribbons attached.

A thirteenth embodiment is shown at 100 in FIG. 23, wherein the ribbons 101 are applied to the stent 102 in a zigzag pattern. As in the previous form, the ribbons can be attached at only one end or at both ends, depending upon the structure of the stent and the ability of the stent to undergo radial expansion with the ribbons attached.

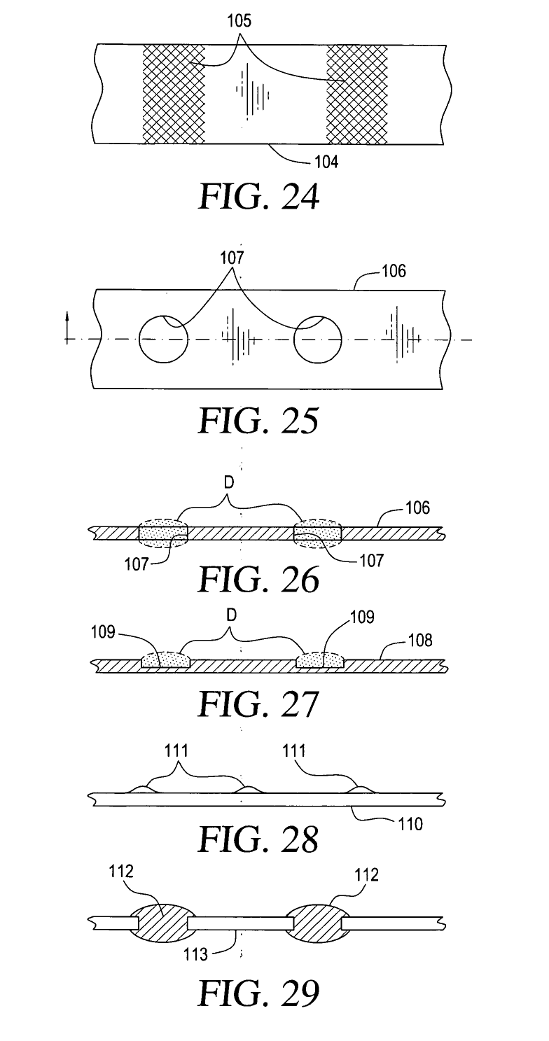

FIG. 24 depicts a ribbon 104 having roughened or textured areas 105 on its surface to provide a surface for enhanced mechanical bonding of a drug or other beneficial agent to the surface of the ribbon, if use of a drug or other agent is desired.

FIGS. 25 and 26 depict a ribbon 106 having openings or holes 107 formed through it to provide a means for applying a drug or other beneficial agent D to the ribbon, if desired.

FIG. 27 depicts a ribbon 108 having recesses or depressions 109 formed in the surface to provide a means for applying a drug or other beneficial agent D to the ribbon, if desired.

FIG. 28 depicts a ribbon 110 having beads 111 of copper or other desired metal deposited on its surface by welding or other suitable means. Beads of copper may also be attached to a conventional stent body such as those shown in FIGS. 1-3, for example.

FIG. 29 depicts an alternate structure, wherein rivets 112 of copper are applied to the ribbon 113.

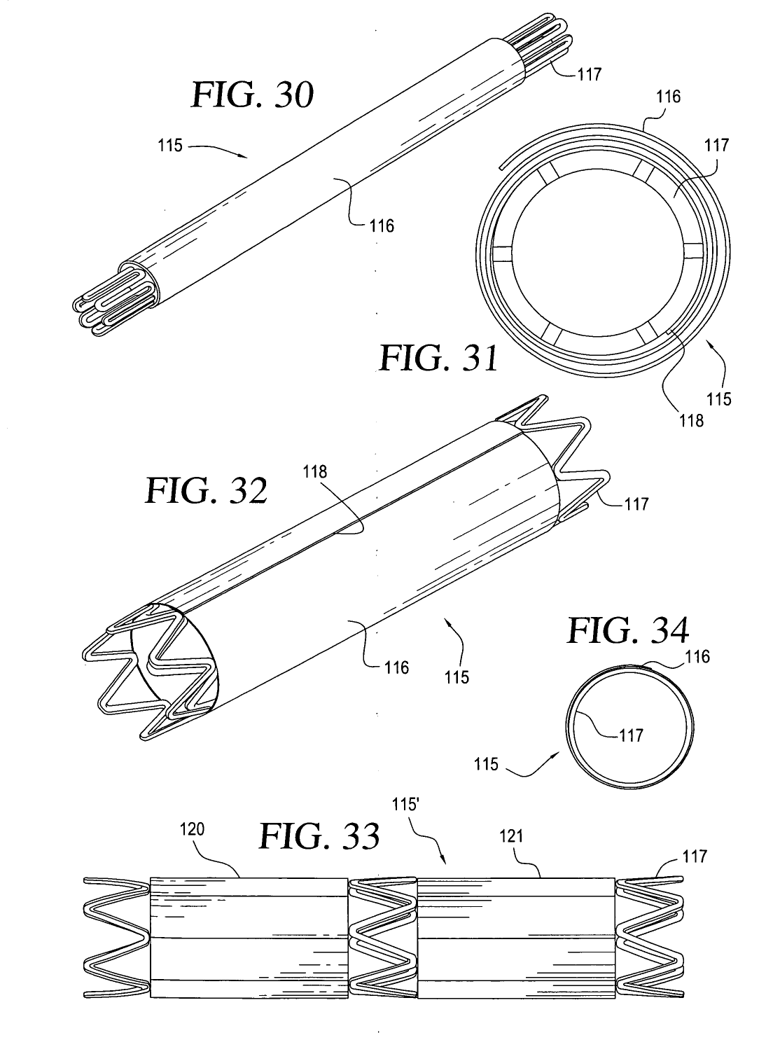

A fourteenth embodiment of a stent with auxiliary structure according to the invention is shown generally at 115 in FIGS. 30-32. In this form, a coiled cover 116 is attached to the stent body 117 along one edge 118 extending longitudinally of the stent. The coiled cover is applied while the stent is in its as-manufactured expanded condition as shown in FIG. 32, after which the stent is collapsed and the cover coiled around it as shown in FIGS. 30 and 31.

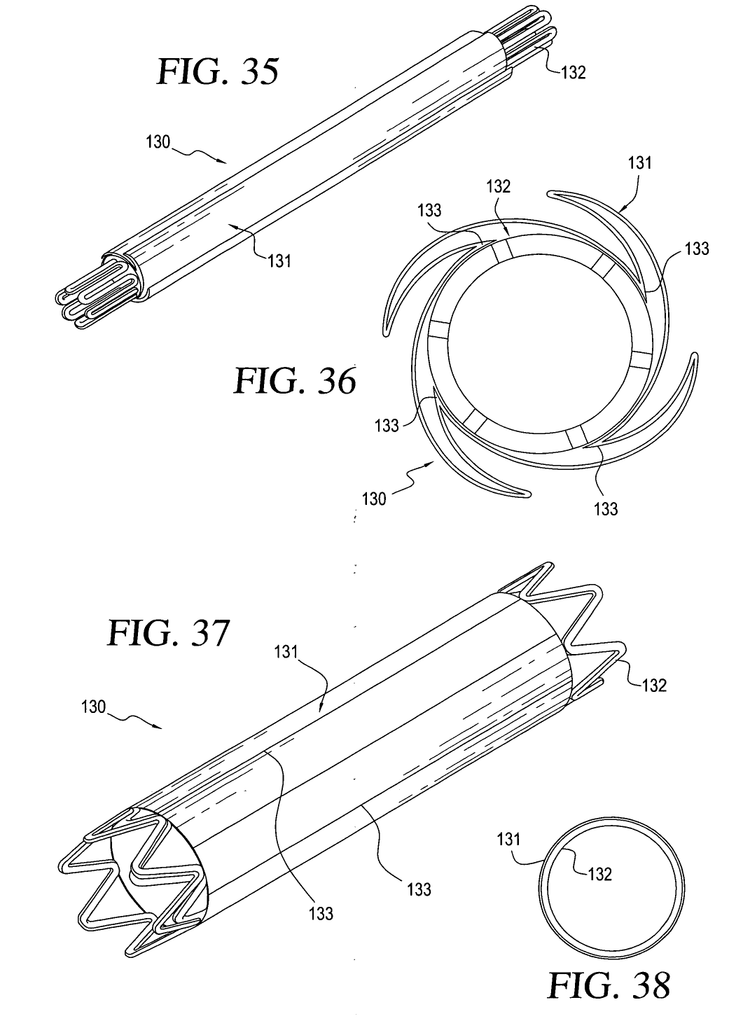

FIG. 33 depicts a variation 115′ of the form of invention shown in FIGS. 30-32, in that a plurality of covers 120 and 121 are applied to the stent body 117 in spaced apart end-to-end relationship along the length of the stent. The covers may be coiled as in FIG. 31, or longitudinally pleated as in FIG. 36, described below.

FIG. 34 is an end view of the expanded stent of FIG. 32, depicting how the coiled cover uncoils when the stent is expanded.

A fifteenth embodiment of the invention is indicated generally at 130 in FIGS. 35-38. In this form, a longitudinally pleated cover 131 is attached to a stent body 132 at longitudinally extending, circumferentially spaced points 133 (see FIG. 36). The stent body itself may be of any suitable conventional construction. The cover is applied to the stent body while the stent is in its as-manufactured expanded condition (see FIG. 37), and is attached by welding or other suitable fastening means. The stent and cover are then collapsed to a contracted condition as shown in FIGS. 35 and 36.

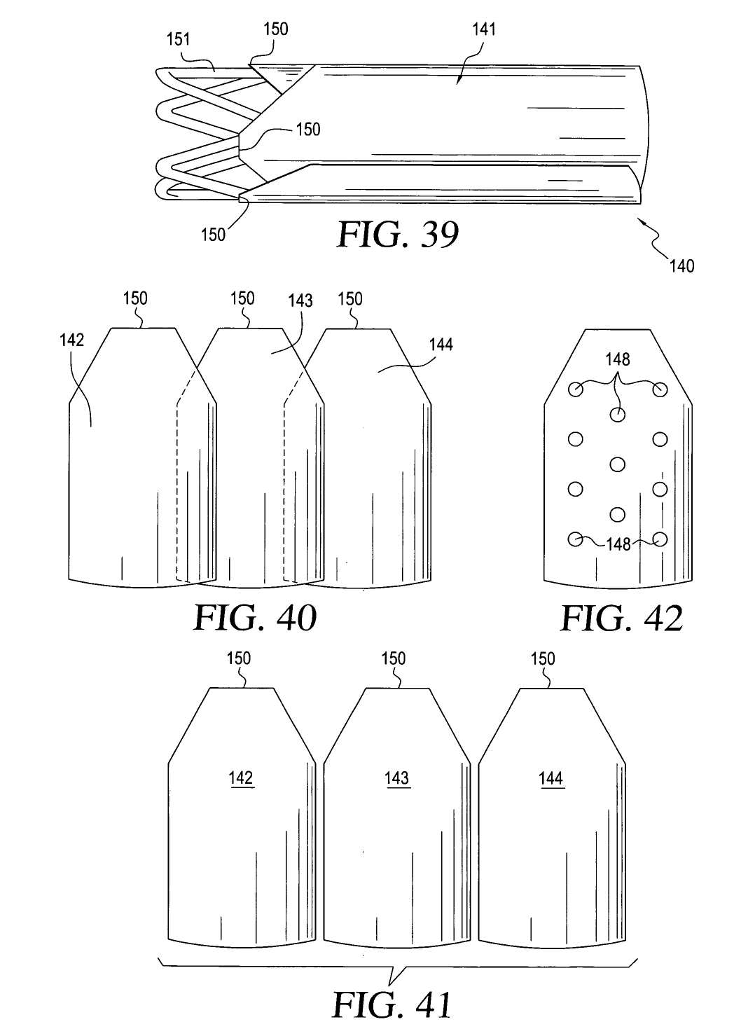

A sixteenth embodiment of the invention is indicated generally at 140 in FIGS. 39-42. In this embodiment, the cover 141 comprises a plurality of overlapping plates 142, 143, 144, etc., fixed by any suitable means, such as, by welding, at an upstream end 150 to the stent body 151 and left unattached over the rest of their length. The plates are attached to the stent body while the stent is in its expanded, as-manufactured condition, at which time the plates 142, 143, 144 preferably will not be overlapping, as depicted in FIG. 41. After the plates are attached, the stent and cover are collapsed to their contracted condition as depicted in FIGS. 39 and 40. Some or all of the plates may be suitably treated, as by texturizing their surface (not shown), or providing depressions or holes 148 therein (FIG. 42), or providing a polymer coating, to hold a drug or drugs applied to the plates, if desired, to achieve the objectives of the present invention.

While particular embodiments of the invention have been illustrated and described in detail herein, it should be understood that various changes and modifications may be made in the invention without departing from the spirit and intent of the invention as defined by the appended claims.

What is claimed is:

1. A stent for implantation into a treatment site in a body lumen, wherein the stent comprises an elongate, open-ended tubular stent body movable from a collapsed position for insertion into a body lumen, to a radially expanded position engaged against an inner surface of the body lumen, and wherein at least a portion of said stent is made from a material selected for its therapeutic properties, said therapeutic properties including at least one of anti-clotting and antimicrobial properties.

2. A stent as claimed in claim 1, wherein:
said stent includes at least one attachment affixed on its outer surface, said attachment made from said material.

3. A stent as claimed in claim 1, wherein:
at least a portion of the stent body is made from said material.

4. A stent as claimed in claim 2, wherein:
the material comprises one of copper and silver.

5. A stent as claimed in claim 3, wherein:
the material comprises one of copper and silver.

6. A stent as claimed in claim 2, wherein:
said at least one attachment comprises a plurality of bands or ribbons extending longitudinally of the stent

7. A stent as claimed in claim 6, wherein:
said at least one attachment comprises a plurality of overlapping plates.

8. A stent as claimed in claim 6, wherein:
the stent body comprises a plurality of interconnected struts forming a lattice-like structure defining a plurality of openings; and
said at least one attachment comprises a separate cover carried on an outer surface of the tubular stent body, covering said plurality of openings and being expandable and contractible with the stent body, said cover being made of said material.

9. A stent as claimed in claim 8, wherein:
said cover comprises a longitudinally pleated structure.

10. A stent as claimed in claim 8, wherein:
said cover comprises a member coiled around the stent body.

11. A stent as claimed in claim 2, wherein:
said at least one attachment comprises a plurality of bands spirally wound around the stent body.

12. A stent as claimed in claim 3, wherein:
said stent body comprises a woven structure of generally longitudinally extending elements and generally transversely extending elements, at least some of said elements formed of said material.

13. A stent as claimed in claim 12, wherein:
some of said elements are made of copper and some are made of silver.

14. A stent as claimed in claim 3, wherein:
said stent body comprises a plurality of closely spaced spirally wound bands that impart a swirling motion to blood flowing through the stent.

15. A stent as claimed in claim 1, wherein:
an inlet end of the stent body is slightly outwardly flared to smooth flow of blood entering the stent.

16. A stent as claimed in claim 1, wherein:
the stent is made of different materials at different portions thereof so that different materials are exposed to body tissue at different locations on the stent.

17. A stent as claimed in claim 16, wherein:
a plurality of rings of dissimilar materials are stacked and secured together to form said tubular stent body so that different materials are exposed to body tissue at different places along the length of the stent, at least some of said rings comprising copper.

18. A stent as claimed in claim 16, wherein:
the stent body comprises concentric layers or tubes of different materials laminated together, at least some of said layers comprising copper.

19. A stent as claimed in claim 8, wherein:
at least one of the layers is formed of strips or panels of different materials arranged side-by-side and extending the length of the stent.

20. A stent as claimed in claim 1, wherein:
said stent body comprises a plurality of strut elements connected to form an open lattice-like structure, said lattice-like structure being formed of different materials in different sections along its length.
